(19) United States
(12) Patent Application Publication
(10) Pub. No.: US 2011/0028406 A1
(43) Pub. Date: Feb. 3, 2011

(54) Pharmaceutical Compositions Comprising A Cyclosporin, A Hydrophilic Surfactant and a Lipophilic Surfactant

(76) Inventor: Bernard Charles Sherman, Toronto (CA)

Correspondence Address: McKinnons Patents Inc, 200 North Service Road W, Unit #1, Suite #303, Oakville, ON, L6M2Y1, CA

(21) Appl. No.: 12/903,859

(22) Filed: Oct. 13, 2010

Related U.S. Application Data
(63) Continuation of application No. 10/499,709, filed on Feb. 14, 2005, now abandoned.

(30) Foreign Application Priority Data
Dec. 20, 2001 (NZ) 516269
Jun. 28, 2002 (NZ) 519837

(51) Int. Cl. A61K 38/13; A61P 37/06

(52) U.S. Cl. 514/20.5

(57) ABSTRACT

Pharmaceutical compositions, which enable high absorption when administered orally, and which comprise a cyclosporin or cyclosporin derivative dissolved in a solvent-surfactant system further comprising a hydrophilic surfactant and a lipophilic surfactant, with minimal quantities of solvents.

This application is a continuation application of U.S. Ser. No. 10/449,709, filed 14 Feb. 2005, which is a US national phase of international application PCT/CA02/01968, filed 19 Dec. 2002, which designated the US and claims priority to NZ Application No. 516269, filed 20 Dec. 2001 and NZ Application No. 519837, filed 28 Jun. 2002. The entire contents of these applications are herein incorporated by reference.

This invention relates to a modification of the invention described and claimed in New Zealand Patent Specification No. 516269.

### TECHNICAL FIELD

This invention is directed to pharmaceutical compositions, which facilitate the administration of cyclosporins and their derivatives of cyclosporins, particularly for oral ingestion.

### BACKGROUND ART

The term “cyclosporin” will be understood to mean any member of the class of nonpolar cyclic oligopeptides with immunosuppressant activity, known as cyclosporins, as defined in the Merck Index, Thirteenth Edition. One such cyclosporin is cyclosporin A, also known as “cyclosporine” and herein referred to as “cyclosporine”. Compositions comprising cyclosporine are sold in the United States and elsewhere under the tradenames Sandimmune™ and Neoral™.

A “derivative” of a cyclosporin will be understood to mean any compound with immunosuppressant activity obtained by modification of a cyclosporin. One example is the compound known as ISA_{TX} 247, which is disclosed in Canadian patent 2,298,572 and specifically claimed in claim 6.

Cyclosporins and their derivatives of cyclosporins are hydrophobic and have low solubility in aqueous media. This makes it difficult to design compositions which exhibit satisfactory absorption into systemic circulation after oral administration.

Cyclosporins and their derivatives of cyclosporins can be dissolved in an organic solvent (e.g. ethanol or propylene glycol), but, if the solvent is water-soluble, when the composition is mixed with gastrointestinal fluid, the cyclosporin or derivative of a cyclosporin will tend to precipitate into particles which are not absorbed.

There are various means disclosed in the prior art for overcoming the tendency to precipitate by use of one or more surfactants.

The first commercially available composition comprising a cyclosporin or derivative of a cyclosporin thereof was an injectable product sold under the tradename Sandimmune™ and comprising, per mL, 50 mg cyclosporine, 650 mg polyoxyl 35 castor oil as a surfactant, with the balance being ethanol as solvent. When this composition is added to water for dilution prior to injectable administration, the composition disperses and the surfactant causes the cyclosporine to be dissolved in a micellar solution, which is sufficiently stable for administration, but from which there may be a small amount of precipitation after some hours.

The composition sold as injectable Sandimmune™ is not suitable for chronic oral administration, because its concentration of cyclosporine is only 50 mg per mL, and because the relatively large amount of both the surfactant and the ethanol content gives rise to toxicity concerns.

The prior art also discloses various means of overcoming these problems to enable workable compositions for oral administration.

U.S. Pat. No. 4,388,307 discloses compositions comprising cyclosporine in an emulsion preconcentrate that forms an emulsion upon being mixed into water. Commercial products sold under the tradename Sandimmune™ for oral administration are made, according to U.S. Pat. No. 4,388,307, and, comprise cyclosporine dissolved in a solvent system comprising ethanol, a vegetable oil and a surfactant. Sandimmune™ for oral administration is or was available as both a liquid preconcentrate and liquid-filled soft-gelatin capsules. Although these compositions enable oral administration, they exhibit absorption that is less than the maximum possible and is variable.

U.S. Pat. No. 5,741,512 discloses emulsion preconcentrates that are similar to the ones disclosed in U.S. Pat. No. 4,888,307 in that they contain, in addition to cyclosporine, a hydrophilic solvent, a lipophilic solvent and a surfactant, but are improved, in that, when added to water, they disperse into droplets of very small size (less than 2000 Angstroms or 0.2 microns), thus leading to improved absorption. These improved emulsion preconcentrates are referred to as microemulsion preconcentrates.

In the U.S. Pat. No. 5,741,512, at Column 8, lines 55 to 60, it is stated that the lipophilic solvent may be any pharmaceutically acceptable solvent which is non-miscible with the hydrophilic solvent, and devoid or substantially devoid of surfactant function. Thus the lipophilic solvent is limited to components that are lipophilic but not amphiphilic (i.e. not both lipophilic and hydrophilic). A component that is amphiphilic is considered to be a surfactant component.

Commercial products made in accordance with the teaching of U.S. Pat. No. 5,741,512 are sold under the tradename Neoral™ as both a liquid preconcentrate of concentration 100 mg/mL, and as liquid-filled soft gelatin capsules.

Neoral™ capsules do enable increased absorption on oral administration relative to Sandimmune™ capsules. However, Neoral™ capsules are still unsatisfactory in the following two respects:

1. The concentration of the cyclosporine in the solution contained with the capsules is only about 10 to 12 percent by weight, so that capsules comprising 100 mg of cyclosporine are quite large and difficult to swallow.

2. The solution contained in the capsules comprises ethanol as a hydrophilic component. Ethanol is volatile, so that the capsules must be packaged individually in metal pouches.

This composition of Neoral™ capsules suggests that the teaching of U.S. Pat. No. 5,741,512 may not enable workable compositions (i.e. compositions that are stable and enable high absorption) that have concentration above about 10 to 12 percent and are ethanol free.

The prior art discloses three possible approaches to achieving water-dispersible cyclosporine preconcentrates that enable high absorption, comparable to that of Neoral™ products, but that are not within the scope of the claims of U.S. Pat. No. 5,741,512.

These approaches are as follows:

1. Omit the hydrophilic solvent, so that the composition comprises cyclosporine dissolved in only lipophilic solvent and surfactant.

2. Omit the lipophilic solvent and use only hydrophilic solvent and hydrophilic (i.e. water-soluble) surfactant. There will thus be no component (other than the cyclosporine) that is not water-soluble. When such a composition is dispersed in water, what is formed is not an emulsion or microemulsion, but either a micellar solution, or a colloidal dispersion.

3. Omit the lipophilic solvent, and use only hydrophilic solvent and one or more surfactants, wherein at least one surfactant is lipophilic (i.e. not water soluble). With appropriately selected solvents and surfactants, such compositions will disperse in water into emulsions or microemulsions, similar to compositions of U.S. Pat. No. 5,741,512.

Compositions in accordance with the first of these three approaches are disclosed in U.S. Pat. No. 5,858,401; and capsules made according to the teaching of this patent are sold in the U.S. market by Sidmak Laboratories Inc. These capsules are bioequivalent to Neoral™ capsules; that is to say, the rate and extent of absorption of the cyclosporine upon oral administration is comparable to that of Neoral™. However, the concentration of the cyclosporine in the solution is again only about 10 to 12 percent by weight, so capsules comprising 100 mg of cyclosporine are relatively large and difficult to swallow, as is the case with Neoral™.

Prior art patents which disclose compositions following the second approach, include U.S. Pat. No. 5,834,017, and U.S. Pat. No. 5,798,333.

A solution made in accordance with the teaching of U.S. Pat. No. 5,834,017 has been sold in the United States and elsewhere under the tradename Sang-Cya™. As is the case with Neoral™, the concentration of cyclosporine in Sang-Cya™ solution is again only about 10 to 12 percent by weight, and the solution also comprises ethanol. Hence, it appears that the teaching of U.S. Pat. No. 5,834,017 may not enable capsules that are filled with a solution that has concentration substantially above 10 percent by weight or that is ethanol free. Hence, Sang-Cya™ solution does not appear to offer any advantage over Neoral™ products.

Soft gelatin capsules filled with a solution within the scope of the claims of U.S. Pat. No. 5,798,333 are sold in the United States by Eon Labs Manufacturing Inc.

The solution in these capsules comprises d-alpha-tocopheryl polyethylene glycol 1000 succinate and polyoxyl 40 hydrogenated castor oil as surfactants, and ethanol as hydrophilic solvent. As with the other aforementioned marketed products, the concentration of the cyclosporine in the solution in the capsules is only about 10 percent by weight, so that the capsules are again relatively large and difficult to swallow. Also, the ethanol content again requires packaging in metal foil pouches.

There are also prior art compositions that follow the third of these three approaches; i.e. that comprise cyclosporine dissolved in a solvent-surfactant system that does not comprise a lipophilic solvent, and comprises a hydrophilic solvent, and a mixture of surfactants, wherein at least one surfactant is lipophilic.

A composition following this third approach is disclosed in international patent application No. PCT/US96/07155. This patent application discloses compositions comprising cyclosporine dissolved in ethanol as hydrophilic solvent, d-alpha-tocopheryl polyethylene glycol 1000 succinate as hydrophilic surfactant, and propylene glycol laurate as lipophilic surfactant. However, when this composition is dispersed in water, it disperses into droplets that are relatively large. It thus does not form a microemulsion and does not enable absorption equivalent to that of Neoral™.

Compositions following the third approach are also disclosed in U.S. Pat. No. 6,008,192. These compositions comprise cyclosporine dissolved in a solvent-surfactant system that comprises ethanol and propylene glycol as hydrophilic solvents, sorbitan monooleate as lipophilic surfactant, and polyoxyl 35 castor oil as hydrophilic surfactant. However, this patent discloses no example of such a composition that comprises more than 13 percent cyclosporine by weight and none that is ethanol free. Claim 12 specifically limits the cyclosporine content to a maximum of 15 percent and the ethanol content to a minimum of 5 percent. A product made in accordance with the teaching of U.S. Pat. No. 6,008,192 is sold in the United States by Abbott Laboratories under the tradename Gengraf™. This product is sold as gelatin capsules filled with a solution. Because the concentration of the cyclosporine in the solution is only about 13 percent, the 100 mg strength capsules are large and difficult to swallow. More particularly, the 100 mg strength is sold as size 00 two-piece hard gelatin capsules which are filled with a solution, closed and sealed. Also, because ethanol is volatile, the capsules must be individually packaged in metal foil pouches to prevent evaporation of the ethanol.

Compositions following the third approach are also disclosed in South African patent 9,813,596. This patent teaches compositions comprising cyclosporine dissolved in a solvent-surfactant system comprising acetylated monoglycerides along with a hydrophilic solvent and hydrophilic surfactant. This patent describes acetylated monoglycerides as being a lipophilic solvent. However, it has surfactant properties. Hence, according to the definition used in U.S. Pat. No. 5,741,512, acetylated monoglycerides is a surfactant (i.e. a lipophilic surfactant) rather than a “lipophilic component”. Hence, compositions of South African patent 9,813,596 fall within the third approach. However, in every example of this patent, the concentration of cyclosporine is again less than 10 percent by weight.

Compositions following the third approach are also disclosed in U.S. Pat. Nos. 5,945,398, 6,187,747, and 6,008,191 all by the same inventors. These patents teach compositions comprising cyclosporine dissolved in a solvent-surfactant system comprising propylene glycol as hydrophilic solvent, together with at least one lipophilic surfactant and at least one hydrophilic surfactant. There are only two examples which show a cyclosporine concentration above 10 percent by weight, i.e. examples 5 and 6 in U.S. Pat. No. 6,008,191. However, because in both examples there is both a relatively low amount of lipophilic surfactant (only 0.5 or 0.55 part per part cyclosporine by weight) and a relatively high amount of propylene glycol as hydrophilic solvent (1.2 or 1.285 part per part cyclosporine by weight), it is unlikely that the composition of either example would disperse in water to provide small enough droplets as needed for good absorption. U.S. Pat. No. 6,008,191 gives absorption data for only example 10, for which the concentration of cyclosporine by weight is only 10 percent. Moreover, even that data is suspect, as each of the three patents purports to give absorption data for a different composition, and yet the absorption data in the three patents are identical.

Further compositions following the third approach are disclosed in U.S. Pat. No. 6,294,192. This patent relates to microemulsion preconcentrates comprising any of a very large number of compounds as the active ingredient, only one of which is cyclosporine. The only example of a microemulsion preconcentrate comprising cyclosporine at above 10 percent by weight is example 57, wherein the amount of cyclosporine is 19.24 percent by weight. However, this example uses glycofurol as hydrophilic solvent at a level of 1 part per part cyclosporine by weight, but use of glycofurol in compositions for oral use is not permitted by some regulatory agencies worldwide, including the U.S. Food and Drug Administration, because it is not recognized as safe for oral use. Moreover, glycofurol has a boiling point below 100° C. Hence, like ethanol, it is volatile, and capsules comprising glycofurol would have to be packaged in foil pouches to prevent evaporation, as for capsules comprising ethanol.

In light of this prior art, the objective of the present invention is to enable a pharmaceutical composition comprising a cyclosporin or derivative of a cyclosporin thereof dissolved in a solvent-surfactant system that meets the following criteria:

1. The composition will disperse in water to form a fine emulsion or microemulsion so as to enable high absorption upon oral administration; and more particularly, upon dispersion in water at 37° C., all or at least most of the cyclosporin or derivative of a cyclosporin will be in droplets of diameter less than 0.45 micron, and preferably less than 0.22 micron.

2. Every inactive ingredient used in the composition will be one that is safe for oral use.

3. The concentration of the cyclosporin or derivative of a cyclosporin will be above 10 percent by weight, and preferably substantially higher.

4. The composition will be stable against precipitation of the cyclosporin or derivative of a cyclosporin, even on prolonged storage at room temperature.

5. The composition will follow the third approach explained in the foregoing. That is to say, it will comprise a cyclosporin or derivative of a cyclosporin dissolved in a solvent-surfactant system that is free or substantially free of lipophilic solvent, and that comprises one or more surfactants, wherein at least one surfactant is lipophilic, and optionally further comprises a hydrophilic solvent.

6. The composition will preferably be free of ethanol, or any other solvent having a boiling point below 100° C.

### DESCRIPTION OF THE INVENTION

It has been found that optimal compositions with relatively high concentration of cyclosporin or derivative of a cyclosporin can be achieved by use of surfactants in which the cyclosporin or derivative of a cyclosporin is soluble, and by minimizing or eliminating not only the lipophilic solvent, but also the hydrophilic solvent.

Compositions of the present invention comprise a cyclosporin or derivative of a cyclosporin dissolved in a solvent-surfactant, system which further comprises a hydrophilic surfactant, and a lipophilic surfactant, and wherein the solvent-surfactant system is free or substantially free of lipophilic solvent, and which may optionally also comprise a hydrophilic solvent, but will preferably be free of hydrophilic solvent.

With appropriate selection of these ingredients, compositions are produced that have relatively high concentrations of a cyclosporin or derivative of a cyclosporin, that are stable, and that will disperse in water to form fine emulsions or microemulsions. More particularly, upon dispersion in water at 37° C., all or at least most of the cyclosporin or derivative of a cyclosporin will be in droplets of diameter less than 0.45 micron, and more preferably less than 0.22 micron.

The amount of cyclosporin or derivative of a cyclosporin in the compositions of the invention by weight will exceed 10 percent, will preferably exceed 13 percent, will more preferably exceed 15 percent, will even more preferably exceed 20 percent, and will most preferably exceed 25 percent. For this purpose, the weight of the composition will be taken to be the total weight of the cyclosporin or derivative of a cyclosporin, hydrophilic surfactant, lipophilic surfactant and hydrophilic solvent, if any, and will exclude the weight of additional auxiliary excipients, if any.

For the purposes of this disclosure, the term “lipophilic” when describing a solvent will have the same meaning as used in U.S. Pat. No. 5,741,512. That is to say “lipophilic solvent” will mean a pharmaceutically acceptable solvent which is devoid or substantially devoid of surfactant function. The compositions of the present invention will be free or substantially free of lipophilic solvent, by which is meant that the amount of lipophilic solvent will be less than 1 part per part cyclosporin or derivative of a cyclosporin by weight, preferably less than 0.5 part per part cyclosporin or derivative of a cyclosporin by weight, and more preferably less than 0.2 part per part cyclosporin or derivative of a cyclosporin by weight. Most preferably, the composition will be entirely free of lipophilic solvent.

The term “hydrophilic solvent” will mean any solvent, that is water-soluble, and in which the cyclosporin or derivative of a cyclosporin is soluble at a level of at least 1 part cyclosporin or derivative of a cyclosporin per 5 parts solvent by weight at 25° C. The term “water-soluble” is to be understood to mean having a solubility of at least 1 part by weight per 100 parts water at 25° C. Suitable hydrophilic solvents include for example, mono-alcohols such as ethanol and benzyl alcohol, and propylene glycol. The preferred hydrophilic solvents are benzyl alcohol and propylene glycol, and most preferred is benzyl alcohol. The compositions of the present invention will preferably be free of ethanol or any other solvent with a boiling point of under 100° C.

If the composition comprises ethanol, which is not preferred, then the amount of cyclosporin or derivative of a cyclosporin by weight should exceed 15 percent of the total weight of composition. If the composition comprises propylene, glycol but not ethanol, the amount of propylene glycol should be less than 1.2 parts per part cyclosporin or derivative of a cyclosporin by weight, or alternatively the amount of lipophilic surfactant should exceed 0.55 part per part cyclosporin or derivative of a cyclosporin by weight.

If the composition comprises glycofurol as hydrophilic solvent, which again is not preferred, the amount of glycofurol should be less than 1 part per part cyclosporin or derivative of a cyclosporin by weight, or alternatively the amount of cyclosporin or derivative of a cyclosporin by weight should exceed 19.25 percent of the total weight of the composition.

The term “stable” will be understood to mean stable against significant precipitation of the cyclosporin or derivative of a cyclosporin out of solution at room temperature. A “stable” composition will thus be one in which all or substantially all of the cyclosporin or derivative of a cyclosporin is dissolved in a solvent-surfactant system, and for which there is no precipitation of cyclosporin or derivative of a cyclosporin out of the solution when it is stored at room temperature.

It is necessary that the composition be stable to have a marketable product. The primary or only purpose of including a hydrophilic solvent in the composition is to add solvent capacity to keep the cyclosporin or derivative of a cyclosporin dissolved. Since one of the objectives of the invention is to achieve high concentration of the cyclosporin or derivative of a cyclosporin, it is preferable to select as lipophilic surfactant and hydrophilic surfactant compounds that are also good solvents for the cyclosporin or derivative of a cyclosporin, so as to minimize or eliminate the need to add a hydrophilic solvent. The amount of hydrophilic solvent in the composition, per part cyclosporin or derivative of a cyclosporin by weight, will preferably be under 1 part, more preferably under 0.6 part, and most preferably none.

The term “hydrophilic surfactant” will include any surfactant that is water-soluble, including, for example:

i) Reaction products of natural or hydrogenated vegetable oils and ethylene glycol; i.e. polyoxyethylene glycolated natural or hydrogenated vegetable oils; for example, polyoxyethylene glycolated natural or hydrogenated castor oils.

ii) Especially suitable are the compounds listed in the United States Pharmacopoeia and National Formulary as polyoxyl 35 castor oil, and polyoxyl 40 hydrogenated castor oil. Most preferred is polyoxyl 35 castor oil.

iii) Polyoxyethylene-sorbitan-fatty acid esters; e.g. lauryl, palmityl, stearyl and oleyl esters e.g. of the type known and listed in the United States Pharmacopoeia and National Formulary as polysorbates. Especially suitable products of this class for use in the compositions of the present invention are polysorbate 20 and polysorbate 80.

iv) d-alpha-tocopheryl polyethylene glycol 1000 succinate.

The term “lipophilic surfactant” will include any surfactant that is not water-soluble but is dispersible in water, including for example:

a) Trans-esterification products of natural vegetable oil triglycerides and polyalkylene polyols. Various forms of trans-esterification product of this class are known and commercially available under the trade name Labrafilw™. Especially useful as a component of the compositions of the invention is Labrafil M1944 CS™, a transesterification product of kernel oil and polyethylene glycol.

b) Mono-, di- and mono/di-glycerides, especially esterification products of caprylic or capric acid with glycerol. Preferred products of this class are those comprising or consisting mainly or essentially of caprylic/capric acid mono- and di-glycerides, such as are commercially available under the trade name Imwitor™. A particularly suitable product of this class for use in the compositions of the invention is the product Imwitor 742™.

c) Soribitan fatty acid esters, such as sorbitan-monolauyl, -monopalmityl, -monostearyl, -tristearyl, -monooleyl, and -trioleyl esters.

d) Monoglycerides, e.g. glyceryl monooleate and acetylated monoglycerides. It will be understood that acetylated monoglycerides consist of glycerol esterified with fatty acids at one of the three hydroxyl functions, with one or both of the other two hydroxyls replaced by an acetyl moiety. By adjusting the degree of saturation of the monoglycerides and the degree of acetylation, different characteristics are obtained. Fully acetylated monoglycerides prepared from unsaturated monoglycerides are liquids at room temperatures. In this context, the phrase “fully acetylated” is intended to mean having a minimum acetylation of about 96 percent. Fully acetylated monoglycerides are currently available from Eastman Chemical Product Inc. under the designations Myvacet 9-08™ and Myvacet 9-45™. Both are liquids at room temperature, having melting points of 4° C. to 12° C.

e) Propylene glycol di-fatty acid esters, such as propylene glycol dicaprylate, propylene glycol di-laurate, and, propylene glycol caprylic-capric acid diester, known and commercially available under the tradename Miglyol 840™; and propylene glycol mono-fatty acid esters such as propylene glycol monocaprylate and propylene glycol monolaurate. Propylene glycol di-fatty acid esters and mono-fatty acid esters are preferred lipophilic surfactants because they are good solvents for cyclosporins and derivatives of cyclosporins, including in particular cyclosporine, and thus help enable compositions with minimal amounts of hydrophilic solvent. Propylene glycol mono-fatty esters are especially preferred. Most preferred is propylene glycol monolaurate; for example, the product commercially available under the tradename Lauroglycol 90™, sold by Gattefosse, which has over 90 percent monoester content.

The compositions of the invention will preferably comprise the following quantities of surfactants for each part of cyclosporin or derivative of cyclosporin by weight:

Hydrophilic surfactant: from about 0.3 part to about 3.0 parts, and more preferably from about 0.4 part to about 2 parts, and even preferably from about 0.5 part to about 1.5 parts.

Lipophilic surfactant: from about 0.6 part to about 2.5 parts, and more preferably from about 0.8 part to about 2.0 parts.

Compositions in accordance with the invention may also contain other ingredients. For example, the composition may include, in addition to the foregoing, one or more other ingredients that are included as diluents, thickening agents, anti-oxidants, flavouring agents, and so forth.

If it is desired to increase the melting point to ensure that the composition is a solid or semi-solid at room temperature, this may be accomplished by adding a further ingredient with a melting point above room temperature, such as, for example, polyethylene glycol with average molecular weight of above 1000 daltons. Polyethylene glycol with average molecular weights of about 3500 daltons and about 8000 daltons are available under the tradename Carbowax™; i.e. Carbowax™ 3500 and Carbowax™ 8000. Such polyethylene glycols are not considered to be within the scope of “hydrophilic solvent” as previously defined, because of the low capacity to dissolve cyclosporine.

The relative proportion of cyclosporin or derivative of a cyclosporin and other ingredients in the compositions of the invention may, of course, vary considerably, depending on the particular ingredients selected. Determination of workable proportions in accordance with the teaching of this specification in any particular instance will generally be within the capability of persons skilled in the art.

Compositions in accordance with the invention will preferably be in the form of solutions that are filled into capsules, such as gelatin capsules, which may be either soft gelatin capsules or two-piece hard gelatin capsules. Two-piece hard gelatin capsules are preferred. Most preferably, the solution used to fill the capsules will comprise an ingredient such as polyethylene glycol with average molecular weight about 1000 daltons to raise the melting point to above 30° C., and preferably above 40° C. The capsules will be filled with the solution as a liquid at elevated temperature. When the filled capsules are filled and cooled to ambient temperature, the contents will solidify to form a solid or semi-solid, so that the capsules will not have to be sealed to prevent leakage.

Empty two-piece hard gelatin capsules are supplied by numerous manufacturers, including Capsugel™. The Capsugel™ cl literature shows that capsules are available in the following standard sizes (among others), with fill capacity in mL as follows:

| Size: | 00 | Oel | 0 | 1 | 2 |
|---|---|---|---|---|---|
| Capacity mL: | 0.95 | 0.78 | 0.68 | 0.50 | 0.37 |

The density of the solutions according to the present invention are about 1.0 g per mL, so that the weight in grams of solution that can be put into a capsule of any size is as approximately equal to the volume in mL. The largest capsule of standard size below size 00 is size Oel, which will hold about 0.8 g.

It thus can be seen that to fill a capsule of a standard size below size 00 with solution containing 100 mg of a cyclosporin or derivative of a cyclosporin requires that the solution have a cyclosporin or derivative of a cyclosporin concentration of about 100/800=12.5 percent by weight, or greater.

Since the present invention enables solutions that produce microemulsions with a cyclosporin or derivative of a cyclosporin concentration well above 10 percent by weight, the present invention enables cyclosporin or derivative of a cyclosporin capsules comprising such a solution and having cyclosporin or derivative of a cyclosporin content of about 100 mg using capsule sizes smaller than size 00; more particularly, the present invention enables capsules containing such a solution and having cyclosporin or derivative of a cyclosporin content of about 100 mg, where the capsule has a fill capacity of less than 0.9 mL, less than 0.8 mL, less than 0.7 mL, less than 0.6 mL, less than 0.5 mL, and even less than 0.4 mL.

The invention will be more fully understood by the following examples which are illustrative but not limiting of compositions in accordance with the present invention.

| EXAMPLE NO. | 1 | 2 | 3 | 4 | 5 | 6 | 7 | 8 | 9 |
|---|---|---|---|---|---|---|---|---|---|
| Cyclosporine | 100 | 100 | 100 | 100 | 100 | 100 | 100 | 100 | 100 |
| Propylene Glycol | 160 | 0 | 0 | 0 | 0 | 0 | 0 | 0 | 0 |
| Benzyl Alcohol | 0 | 80 | 60 | 80 | 70 | 50 | 50 | 0 | 0 |
| Polyoxyl 35 Castor Oil | 220 | 220 | 120 | 200 | 200 | 80 | 60 | 100 | 100 |
| Sorbitan Monooleat | 160 | 160 | 0 | 0 | 0 | 0 | 0 | 0 | 0 |
| Glyceryl Monooleate | 0 | 0 | 120 | 0 | 0 | 0 | 0 | 0 | 0 |
| Labrafil M1944CS ™ | 0 | 0 | 0 | 200 | 0 | 0 | 0 | 0 | 0 |
| Myvacet 9-45 ™ | 0 | 0 | 0 | 0 | 120 | 110 | 110 | 0 | 0 |
| Lauroglycol 90 ™ | 0 | 0 | 0 | 0 | 0 | 0 | 0 | 150 | 210 |
| Polyethylene Glycol | 20 | 20 | 0 | 20 | 20 | 20 | 60 | 10 | 30 |
| 8000 | | | | | | | | | |
| Total | 660 | 580 | 400 | 600 | 510 | 360 | 380 | 360 | 440 |
| Net fill per capsule, mg | 660 | 580 | 400 | 600 | 510 | 360 | 380 | 360 | 440 |
| Capsule size used | 0 | 0 | 1 | 0 | 1 | 2 | 2 | 2 | 1 |

For each of the examples, the ingredients in the proportions shown were mixed at elevated temperature (above 60° C.) until a clear solution was formed. Upon cooling to room temperature, the solution of example 3 remained a clear solution.

For all of the other examples, because of the presence of polyethylene glycol 8000, the solutions solidified to form a semi-solid when cooled to room temperature.

When still hot, some of the solution of each of the examples was filled into two-piece hard gelatin capsules of the sizes and with the net fill per capsule as shown. A capsule of each example was dissolved in 1000 mL of water at 37° C. In each case, when the shell dissolved, the contents of the capsule dispersed into a fine emulsion or microemulsion, and most of the cyclosporine was in droplets of diameter less than 0.22 micron, as determined by filtering the emulsion through a 0.22 micron filter and assaying the filtrate for cyclosporine.

It will be understood that compositions similar to those of examples 1-9 may be made using a cyclosporin or derivative of a cyclosporin other than cyclosporine, and in particular using ISA_{TX} 247.

### INDUSTRIAL APPLICABILITY

From the foregoing description, it will be apparent that the present invention provides improved compositions for the administration and absorption of a cyclosporin or derivative of a cyclosporin.

1. A pharmaceutical composition comprising cyclosporine dissolved in a solvent-surfactant system, which, when dispersed in water at 37° C., forms an emulsion in which more than 50 percent of the cyclosporine is in droplets of diameter of about 0.45 micron or less, and wherein the solvent-surfactant system comprises:
a hydrophilic surfactant selected from: a reaction product of natural or hydrogenated vegetable oil and ethylene glycol; polyoxyl 35 castor oil; polyoxyl 40 hydrogenated castor oil; a polyoxyethylene-sorbitan fatty acid ester; and d-alpha-tocopheryl polyethylene glycol 1000 succinate;
a lipophilic surfactant selected from: a transesterification product of natural vegetable oil and polyalkylene glycol; mono-, di- and mono/di-glycerides; a sorbitan fatty acid ester; sorbitan monooleate; a monoglyceride; glyceryl monooleate; acetylated monoglycerides; a propylene glycol fatty acid ester; a propylene glycol fatty acid monoester and propylene glycol monolaurate; and
benzyl alcohol, with the provisos that:
i) the amount of cyclosporine by weight is greater than 10 percent of the total weight of cyclosporine, the hydrophilic surfactant, the lipophilic surfactant and benzyl alcohol;
ii) the composition is free of lipophilic solvent or comprises less than 1 part by weight lipophilic solvent per part by weight cyclosporine;
iii) the amount of benzyl alcohol is less than 1 part by weight per part by weight cyclosporine;
iv) if the compositions comprises ethanol as a hydrophilic solvent, the amount of cyclosporine by weight exceeds 15 percent of the total weight of the cyclosporine, hydrophilic surfactant, lipophilic surfactant and benzyl alcohol;
v) if the composition comprises glycofurol, the glycofurol is present in an amount of less than 1 part by weight per part by weight cyclosporine, or alternatively the cyclosporine is present in an amount by weight of greater than 19.25 percent of the total weight of cyclosporine, hydrophilic surfactant, lipophilic surfactant and benzyl alcohol; and
vi) if the composition comprises propylene glycol and does not comprise ethanol, the propylene glycol is present in an amount of less than 1.2 parts by weight per part by weight cyclosporine, or alternatively the lipophilic surfactant is present in an amount which exceeds 0.55 part by weight per part by weight cyclosporine.

2. The composition of claim 1 that is free or substantially free of lipophilic solvent.

3. The composition of claim 1 wherein the composition is free of any hydrophilic solvent having a boiling point under 100° C.

4. The composition of claim 1 wherein the amount of cyclosporine by weight is greater than 13 percent of the total weight of the cyclosporine, the hydrophilic surfactant, the lipophilic surfactant and benzyl alcohol.

5. The composition of claim 1 wherein the amount of cyclosporine by weight is greater than 25 percent of the total weight of the cyclosporine, the hydrophilic surfactant, the lipophilic surfactant and benzyl alcohol.

6. The composition of claim 1 wherein the amount of benzyl alcohol is less than 0.6 part by weight per part by weight cyclosporine.

7. The composition of claim 1 wherein the amount of lipophilic surfactant is from 0.6 part by weight to 2.5 parts by weight per part by weight cyclosporine.

8. The composition of claim 1 wherein the amount of lipophilic surfactant is from 0.8 part by weight to 2.0 parts by weight per part by weight cyclosporine.

9. The composition of claim 1 wherein the amount of hydrophilic surfactant is from 0.3 part by weight to 3 parts by weight per part by weight cyclosporine.

10. The composition of claim 1 wherein the amount of hydrophilic surfactant is from 0.4 part by weight to 2 parts by weight per part by weight cyclosporine.

11. A capsule for oral administration containing the composition of claims 1.

12. The capsule of claim 11 wherein the amount of cyclosporine is about 100 mg and the fill capacity of the capsule is less than 0.9 mL.

13. The capsule of claim 12 wherein the fill capacity of the capsule is less than 0.8 mL.

14. The capsule of claim 13 wherein the fill capacity of the capsule is less than 0.4 mL.

15. The composition of claim 1 wherein the amount of hydrophilic surfactant is from 0.5 part to 1.5 parts per part cyclosporine by weight.

16. The composition of claim 1 comprising less than 0.5 part lipophilic solvent per part cyclosporine by weight.

17. The composition of claim 1 comprising less than 0.2 part by weight lipophilic solvent per part by weight cyclosporine.

18. The composition of claim 1 which disperses in water at 37° C. to form an emulsion in which more than 50 percent of the cyclosporine is in droplets having a diameter less than 0.22 micron.

19. The composition of claim 1 further comprising polyethylene glycol with average molecular weight above 1000 Daltons.

20. A pharmaceutical composition comprising polyethylene glycol with average molecular weight above 1000 Daltons and cyclosporine dissolved in a solvent-surfactant system, which, when dispersed in water at 37° C., forms an emulsion in which more than 50 percent of the cyclosporine is in droplets of diameter of 0.22 micron or less, and wherein the solvent-surfactant system comprises:
a hydrophilic surfactant in an amount from 0.5 part by weight to 1.5 parts by weight per part by weight cyclosporine, the hydrophilic surfactant selected from: a reaction product of natural or hydrogenated vegetable oil and ethylene glycol; polyoxyl 35 castor oil; polyoxyl 40 hydrogenated castor oil; a polyoxyethylene-sorbitan fatty acid ester; and d-alpha-tocopheryl polyethylene glycol 1000 succinate;
a lipophilic surfactant in an amount from 0.8 part by weight to 2.0 parts by weight per part by weight cyclosporine, the lipophilic surfactant selected from: a transesterification product of natural vegetable oil and polyalkylene glycol; mono-, di- and mono/di-glycerides; a sorbitan fatty acid ester; sorbitan monooleate; a monoglyceride; glyceryl monooleate; acetylated monoglycerides; a propylene glycol fatty acid ester; a propylene glycol fatty acid monoester and propylene glycol monolaurate; and
benzyl alcohol, with the provisos that:
i) the amount of cyclosporine by weight is greater than 25 percent of the total weight of cyclosporine, hydrophilic surfactant, lipophilic surfactant and benzyl alcohol;
ii) the composition is free of lipophilic solvent;
iii) the amount of benzyl alcohol is less than 0.6 part by weight per part by weight cyclosporine;
iv) if the composition comprises ethanol as a hydrophilic solvent, the amount of cyclosporine by weight exceeds 15 percent of the total weight of the cyclosporine, hydrophilic surfactant, lipophilic surfactant and benzyl alcohol;
v) if the composition comprises glycofurol, the glycofurol is present in an amount of less than 1 part by weight per part by weight cyclosporine, or alternatively the cyclosporine is present in an amount by weight of greater than 19.25 percent of the total weight of cyclosporine, hydrophilic surfactant, lipophilic surfactant and benzyl alcohol; and
vi) if the composition comprises propylene glycol and does not comprise ethanol, the propylene glycol is present in an amount of less than 1.2 parts by weight per part by weight cyclosporine, or alternatively the lipophilic surfactant is present in an amount which exceeds 0.55 part by weight per part by weight cyclosporine.
